(19) United States
(12) Patent Application Publication
(10) Pub. No.: US 2016/0375206 A1
(43) Pub. Date: Dec. 29, 2016

(54) MOUTHPIECE FOR AN INHALER DEVICE

(71) Applicant: Pari GmbH, Starnberg (DE)

(72) Inventors: Jens Gramann, Grafelfing (DE); Yella Wahner, Munich (DE)

(73) Assignee: Pari GmbH, Starnberg (DE)

(21) Appl. No.: 15/188,216

(22) Filed: Jun. 21, 2016

(30) Foreign Application Priority Data
Jun. 26, 2015 (DE) 10 2015 211 931.9

(51) Int. Cl. A61M 15/00; A61M 11/02; A61M 11/06; A61M 11/00

(52) U.S. Cl. CPC A61M 15/0021; A61M 11/005; A61M 11/02; A61M 11/06; A61M 15/0086; A61M 15/009; A61M 2205/586; A61M 2202/064

(57) ABSTRACT

Mouthpiece (1) for an inhaler device having an inlet, which is connectable to an inhaler device, an outlet and a holding device (2).

The invention relates to a mouthpiece for an inhaler device.

Mouthpieces for inhaler devices are known in the prior art and are described, for example, in EP 0 626 180 B1.

Some inhaler devices can be operated hands-free with such a mouthpiece. In this case, however, a user must either bite very firmly or additionally stabilize the mouthpiece using the lower lip to prevent the inhaler device from tilting. This is uncomfortable and tiring for the user. If the user bites very firmly, the mouthpiece may be damaged.

The object of the invention is therefore to provide a mouthpiece with which an inhaler device can be operated more comfortably hands-free.

The object is achieved by a mouthpiece for an inhaler device having an inlet, which is connectable to an inhaler device, an outlet and a holding device.

The inlet is configured to admit aerosol from an inhaler device into the interior of the mouthpiece. The outlet is configured to let aerosol out of the mouthpiece and into the mouth of a user.

The holding device is expediently configured to fix the mouthpiece with the help of a tooth or a plurality of teeth of the user. The inhaler device can also be held via the mouthpiece. For this purpose, the holding device preferably has support or contact surfaces which are arranged in such a manner that, in conjunction with a tooth or a plurality of teeth of the user, they can prevent a turning or tilting movement and slipping of the mouthpiece. Two support surfaces are expediently provided, said surfaces pointing in different directions and being offset from one another, or being so large that partial areas of the support surfaces provide adequate support for a tooth, and these partial regions are offset from one another.

In one embodiment, the support surfaces are configured to rest against incisors of a user. It is preferable if a support surface is configured to rest on a vestibular surface or an oral surface of a tooth. It is expedient if a first support surface is configured to rest against the oral surface of an incisor of the upper jaw and a second support surface is configured to rest against the vestibular surface of an incisor of the lower jaw. It is expedient if a first support surface is configured to rest against the oral surface of an incisor of the upper or lower jaw and a second support surface is configured to rest against the vestibular surface of the same incisor.

In one embodiment, the support surfaces are configured to cooperate with the occlusal surfaces of molars. The support surfaces are expediently so large that the mouthpiece together with an inhaler device can be comfortably held by clamping between the molars. If two support surfaces are provided on one side of the mouthpiece, the mouthpiece can be made small. The mouthpiece can be held particularly comfortably if two support surfaces in each case are provided on both sides of the mouthpiece. Thus, right and left molars can hold the mouthpiece.

In an advantageous embodiment, a support surface is provided on a notch, recess, elevation, thickening or bead.

The inhaler device is preferably an aerosol generating device. An aerosol generating device preferably has a nebulizer, an atomizer, a humidifier, a compressed air nebulizer, an air atomizer, an electronic nebulizer, an ultrasound nebulizer, an electro-hydrodynamic nebulizer, an electrostatic nebulizer, a membrane nebulizer, a nebulizer with a vibrating membrane, an electronic nebulizer with a vibrating membrane, a mesh nebulizer, a jet nebulizer, an inhaler (MDI), a powder atomizer (DPI) or a combination thereof. In one embodiment, the inhaler has a pressurized canister with a drug and a propellant gas. The canister is expediently connected to a manually operable actuator. It is advantageous if the inhaler is configured to dispense a specific amount of medication in aerosol form when activated. In one embodiment, the aerosol generating device is configured for use with respiratory devices.

It is advantageous if the inhaler device is a device for delivering aerosols. The device for delivering aerosols preferably has a holding chamber, a spacer or a chamber. Devices for delivering aerosols are preferably devices which are provided for use with inhalers (MDIs). They provide medication spaces which are configured to accept aerosol preferably from inhalers so that users can breathe it out therefrom. Spacers have no inhalation and exhalation valves which means that a user should coordinate his breathing so that he does not exhale into the spacer. Chambers or holding chambers have exhalation and also preferably inhalation valves. Thus it can be achieved that an exhalation flow is not directed into the space in which the aerosol is located. It can be achieved that a drug can only get out of the medication space during inhalation.

Aerosols are mixtures of solid or liquid suspended particles and a gas.

Aerosols are preferably provided for application to or in parts of the human or animal body, such as skin, body cavities, body orifices, nose, paranasal sinuses, maxillary sinus, frontal sinus, sphenoid sinus, ethmoid sinuses, pharynx, larynx, trachea, lung, main bronchus, bronchi, bronchioles, pulmonary alveoli, joints or abdominal cavity. Aerosols can be used to diagnose, prevent, treat human and animal diseases or to immunize humans or animal against diseases.

In one embodiment, the holding device has an upper form fitting device and a lower form fitting device. Thus a tilting moment can be distributed between upper and lower jaw such that it is particularly comfortable to hold the inhaler device. The upper form fitting device is preferably configured to cooperate with a tooth of an upper jaw. The lower form fitting device is preferably configured to cooperate with a tooth of a lower jaw. If the upper form fitting device and the lower form fitting device are offset in relation to one another, they can cooperate with the teeth of the user in such a way that the user can adopt a particularly comfortable jaw position.

The upper form fitting device preferably has a rib or a channel. Thus the upper form fitting device can be provided in a simple manner.

In one embodiment, the lower form fitting device has two ribs or one channel. Thus the lower form fitting device can be provided in a simple manner.

It is advantageous if the holding device has a plurality of lower form fitting devices. As a result, users with different jaw positions can comfortably use the mouthpiece. The user can then use the lower form fitting device that best fits his teeth. Users can also switch between the various lower form fitting devices to vary the load on the muscles. A plurality of depressions, for example channels or grooves, or elevations, for example beads or ribs, are expediently arranged in parallel.

In one embodiment, the holding device has a plurality of upper form fitting devices. As a result, users can vary the position of the mouthpiece such that it extends more or less into the mouth. The user expediently selects a position of the mouthpiece which makes it possible to direct as much aerosol as possible into the respiratory tract and to deposit as little aerosol as possible in the oral cavity. In one embodiment, a plurality of recesses or elevations are provided in parallel.

In one embodiment, the holding device includes polypropylene. The holding device can be provided in a particularly simple manner if the mouthpiece is manufactured together with the holding device as an injection molding. The injection molding preferably includes polypropylene. In one embodiment, the injection molding consists essentially of polypropylene. It is advantageous if the holding device includes a soft material. It is expedient if the mouthpiece includes a soft material adjacent to an outlet side. The soft material is expediently softer than another material which is included in the mouthpiece. In one embodiment, the soft material is a thermoplastic elastomer. The soft material preferably has a Shore hardness of 20 ShA to 99 ShA, especially preferably of 70 ShA to 90 ShA. The soft material is expediently provided on a contact or support surface in such a manner that a tooth of the user can rest against the soft material. This can increase the comfort for a user. In one embodiment, recesses, such as channels, or elevations, such as beads or ribs, are coated with a soft material.

Advantages can be achieved if the holding device includes a hard material. It is particularly advantageous if the mouthpiece includes a hard material adjacent to an outlet side. The hard material has a greater hardness than another material of the mouthpiece. The hard material preferably has a Shore hardness of 30 ShD to 90 ShD, especially preferably of 76 ShD to 89 ShD. The hard material is expediently provided on a contact or support surface in such a manner that a tooth of the user can rest on the hard material. In one embodiment, recesses, such as channels, or elevations, such as beads or ribs, are coated with a hard material. By providing the hard material, it is possible to provide a holding device that has particularly good durability.

A multi-component injection molding expediently includes the holding device.

In one embodiment, the flow cross-section of the mouthpiece is not deformable in use. It is expedient if the mouthpiece includes a non-elastic material. The modulus of elasticity of the non-elastic material is preferably between 0.5 and 1000 kN/mm², especially preferably between 5 and 400 kN/mm².

The object is also achieved by a mouthpiece for an inhalation therapy unit having an inhaler device and a mouthpiece, which has an inlet that is connectable to an inhaler device, an outlet and a holding device.

The invention is described in greater detail below on the basis of embodiments with reference to the associated Figures.

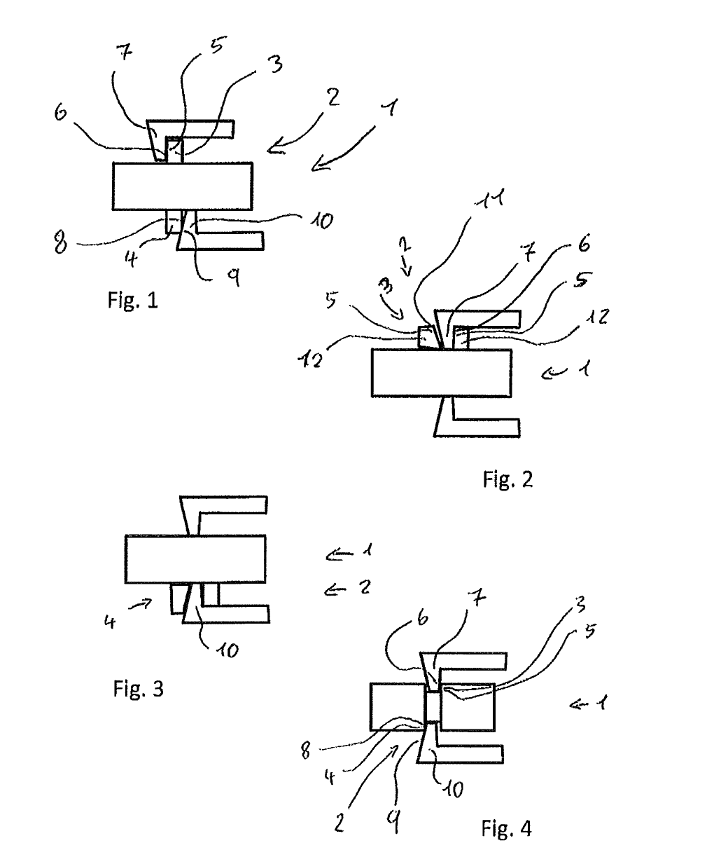

FIG. 1 shows a mouthpiece with a holding device.

FIG. 2 shows a further mouthpiece with a holding device.

FIG. 3 also shows a mouthpiece with a holding device.

FIG. 4 shows a mouthpiece with a holding device in which the upper and lower form fitting devices are designed as a groove.

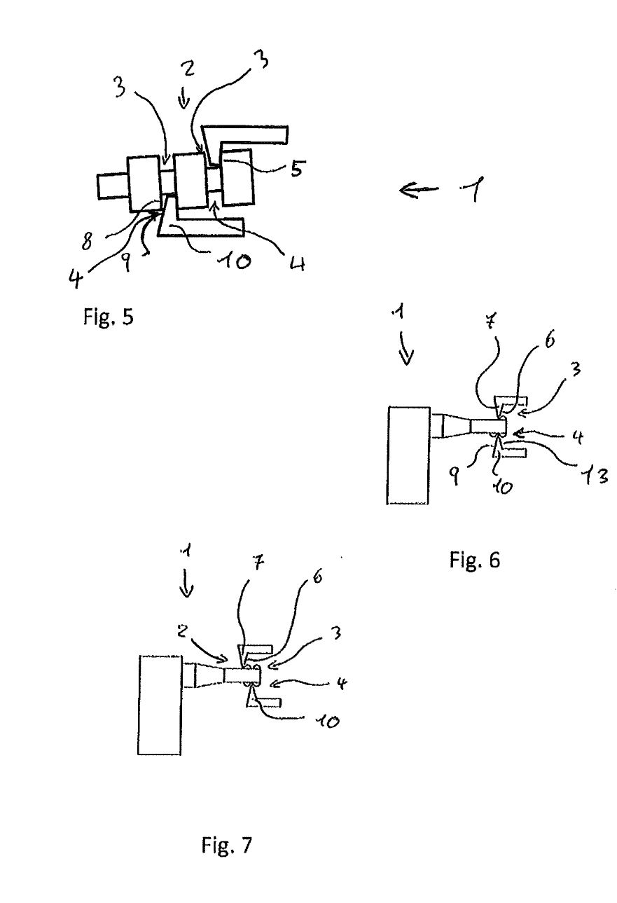

FIG. 5 also shows a mouthpiece with a holding device. Two upper form fitting devices and two lower form fitting devices are present, each having a groove.

FIG. 6 shows a holding device which has an upper form fitting device with one rib and a lower form fitting device with two ribs.

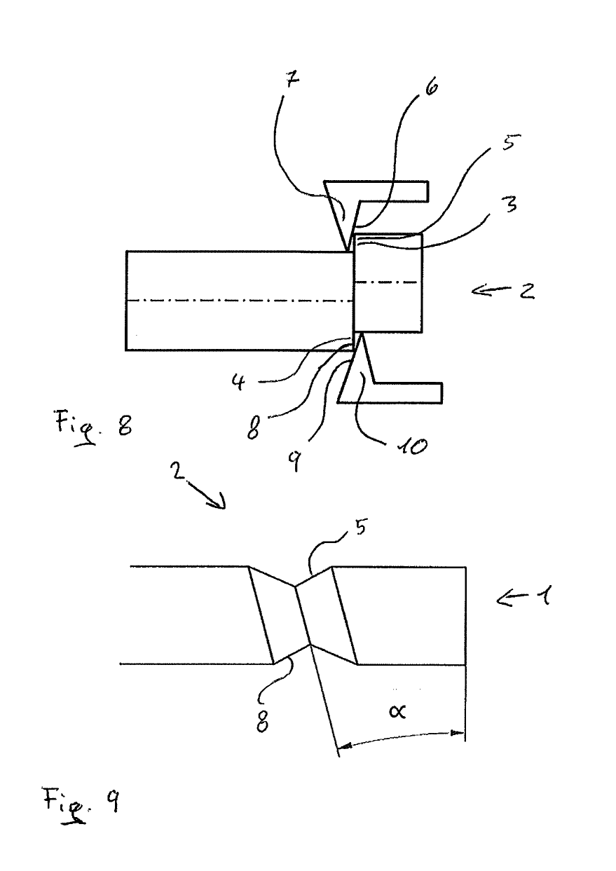

FIG. 7 shows a holding device which has two upper form fitting devices each with one rib and one lower form fitting device with two ribs.

FIG. 8 shows a holding device.

FIG. 9 shows a holding device which is designed in the shape of two truncated cones.

FIGS. 10 and 11 show a mouthpiece which has a holding device with one upper form fitting device and one lower form fitting device.

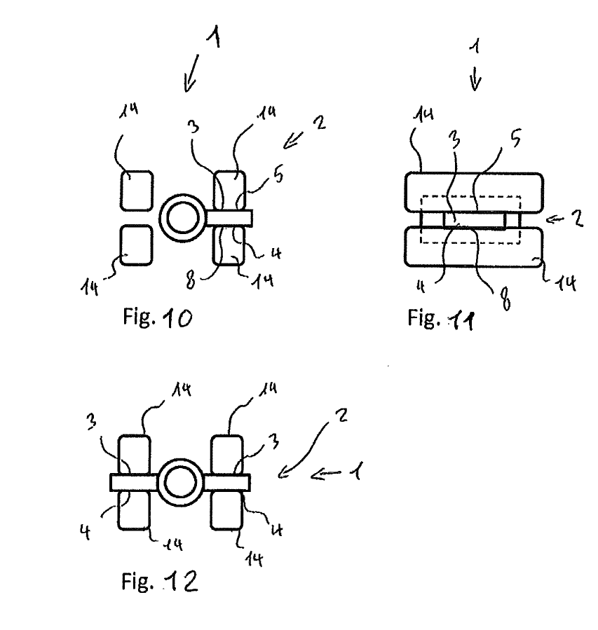

FIG. 12 shows a mouthpiece which has a holding device with two upper form fitting devices and two lower form fitting devices.

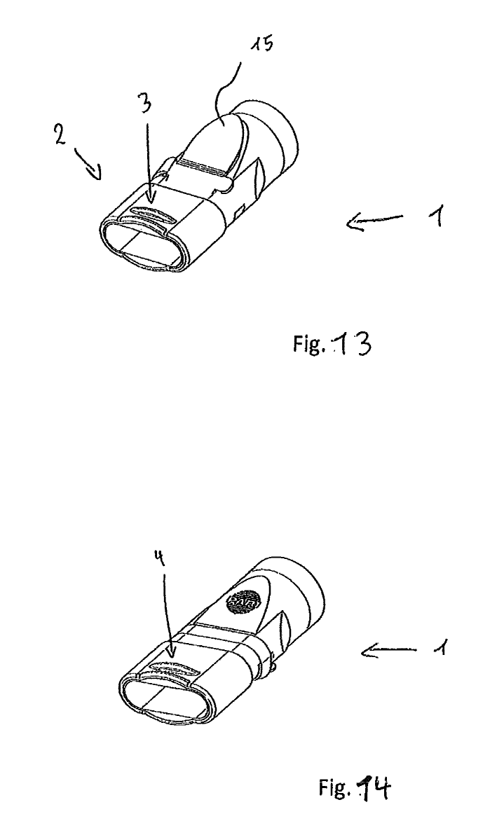

FIGS. 13 and 14 show the embodiment of a mouthpiece with holding device.

FIG. 1 shows a mouthpiece 1 with a holding device 2. The holding device 2 has an upper form fitting device 3 in the shape of a rib and a lower form fitting device 4 also in the shape of a rib. The upper form fitting device 3 is provided in a region of the mouthpiece 1 which is at the top when the mouthpiece 1 is being used, the lower form fitting device 4 is provided in a region of the mouthpiece 1 which is at the bottom when the mouthpiece 1 is being used.

A upper support surface 5 is present on the upper form fitting device 3. The upper support surface 5 is provided in a region of the upper form fitting device 3 which points out of the mouth of the user when the mouthpiece 1 is being used. When in use, the oral surface 6 of an upper incisor 7 rests against the upper support surface 5.

A lower support surface 8 is present on the lower form fitting device 4. The lower support surface 8 is provided in a region of the lower form fitting device 4 which points into the mouth of the user when the mouthpiece 1 is being used. When in use, the vestibular surface 9 of a lower incisor 10 rests against the lower support surface 8.

The holding device 2 can take the weight of a mouthpiece 1 and an inhaler device. It is easily possible to prevent tilting of the mouthpiece 1 and inhaler device as the mouthpiece 1 can be supported inside on the upper incisor 7 via the upper support surface 5 and outside on the lower incisor 10 via the lower support surface 8.

FIG. 2 shows a mouthpiece 1 with a holding device 2. The holding device 2 has an upper form fitting device 3 in the shape of two elevations 12. Two upper support surfaces 5 are present on the upper form fitting device 3. The upper support surfaces 5 are arranged in such a way that the first upper support surface 5 is configured to rest against the outside 11 of an upper incisor 7 and the second upper support surface 5 is configured to rest against the oral surface 6 of the upper incisor 7. For this, the two upper support surfaces 5 oppose each other. They are aligned with each other. The upper support surfaces 5 are so large that tilting or slipping of the mouthpiece 1 can comfortably be prevented. On tilting of the mouthpiece 1, an upper region of the second upper support surface 5, which is located on the oral surface 6 of the upper incisor 7, is supported from the inside against the upper incisor 7. A lower region of the first upper support surface 5, which is located on the vestibular surface 11 of the upper incisor 7, is supported from the outside against the upper incisor 7.

FIG. 3 also shows a mouthpiece 1 with a holding device 2. It is constructed similarly to the mouthpiece 1 shown in FIG. 2 but instead of the upper form fitting device 3, a lower form fitting device 4 is provided which is configured to hold the mouthpiece 1 on a lower incisor 10.

FIG. 4 shows a mouthpiece 1 with a holding device 2 in which the upper and lower form fitting devices 3, 4 are designed as a groove. In the embodiment shown here they are designed jointly as a peripheral groove. Support surfaces 5, 8 of the form fitting devices 3, 4 are provided on the side surfaces of the groove. The upper support surface 5 of the upper form fitting device 3 is provided on the groove side which is closer to the inside of the mouth and when in use rests against the oral surface 6 of an upper incisor 7. The lower support surface 8 of the lower form fitting device 4 is provided on the groove side which is further away from the inside of the mouth and when in use rests against the vestibular surface 9 of a lower incisor 10. As a result, turning and slipping of the mouthpiece 1 can be prevented according to the embodiment shown in FIG. 1.

FIG. 5 also shows a mouthpiece 1 with a holding device 2. Two upper form fitting devices 3 and two lower form fitting devices 4 are present, each having a groove. Support surfaces 5, 8 of the form fitting devices 3, 4 are each provided on the side surfaces of a groove.

The holding device 2 shown in FIG. 5 resembles the holding device 2 shown in FIG. 4. As a result of the fact that a plurality of upper and lower form fitting devices 3, 4 are available, a user of the mouthpiece 1 can select form fitting devices 3, 4 which fit well with his teeth. As the teeth of the users vary widely, it is possible in this way for a mouthpiece to be used comfortably by many different users.

The holding device 2 shown in FIG. 6 has an upper form fitting device 3 with one rib and a lower form fitting device 4 with two ribs. During use of the mouthpiece 1, the upper form fitting device 3 rests against the oral surface 6 of an upper incisor 7 with a support surface and can thus prevent the mouthpiece 1 from slipping.

A lower incisor 10 can cooperate with the lower form fitting device 4 in such a manner that both a tilting moment can be absorbed and also slipping of the mouthpiece 1 can be prevented. The tilting moment can be particularly well absorbed as one rib can be supported on the vestibular surface 9 of a lower incisor 10. Slipping can be particularly well prevented as the rib of the upper form fitting device 3 can be supported on the oral surface 6 of an upper incisor 7 in case of slipping. A rib of the lower form fitting device 4 can be supported on the oral surface 13 of a lower incisor 10 in case of slipping.

The holding device 2 shown in FIG. 7 has two upper form fitting devices 3 each with one rib and one lower form fitting device 4 with two ribs. A user can select which of the upper form fitting devices 3 should rest against the oral surface 6 of an upper incisor 7 when using the mouthpiece 1. As a result, the holding device 2 can be used comfortably by users with different positional relationship of the maxillary anterior teeth to the mandibular anterior teeth. As in the embodiment shown in FIG. 6, a lower incisor 10 can cooperate with the lower form fitting device 4 in such a manner that both a tilting moment can be absorbed and also slipping of the mouthpiece 1 can be prevented.

FIG. 8 shows a holding device 2 in which the upper form fitting device 3 has a support surface 5 which is configured to rest against the oral surface 6 of an upper incisor 7.

The lower form fitting device 4 shown in FIG. 8 has a lower support surface 8 which is configured to rest against the vestibular surface 9 of a lower incisor 10.

The holding device 2, like the holding device 2 shown in FIG. 1, can take the weight of a mouthpiece 1 and an inhaler device. It is also easily possible to prevent tilting of the mouthpiece 1 and inhaler device as the mouthpiece 1 can be supported inside on the upper incisor 7 via the upper support surface 5 and outside on the lower incisor 10 via the lower support surface 8.

The upper support surface 5 and the lower support surface 8 are formed at the offset point of a body. In FIG. 8, the body has a cylindrical shape. The upper support surface 5 and the lower support surface 8 are therefore delimited by two circles of the same diameter which are displaced relative to one another.

FIG. 9 shows a holding device 2 which is designed in the shape of two truncated cones in such a manner that a peripheral groove with sloping side surfaces is present. The truncated cones are at an angle to a longitudinal axis of the mouthpiece 1. The angle between the longitudinal axes of the truncated cones and the longitudinal axis of the mouthpiece 1 is 15°. The upper support surface 5 and the lower support surface 8 are each formed by a sloping side surface. The upper support surface 5 is suitable to rest against the oral surface 6 of an upper incisor not shown in the figure. The lower support surface 8 is suitable to rest against the vestibular surface of a lower incisor not shown in the figure. As a result, the mouthpiece 1 can be held and tilting prevented.

The mouthpiece 1 shown in FIGS. 10 and 11 also has a holding device 2 with one upper form fitting device 3 and one lower form fitting device 4. FIG. 10 shows a section through a mouthpiece 1 with holding device 2 and four molars 14. FIG. 11 shows a lateral view of the mouthpiece 1 shown in FIG. 10. In the embodiment shown in FIGS. 10 and 11, the holding device 2 is designed to cooperate with molars 14 of a user. The upper form fitting device 3 has one upper support surface 5 which points upwards when the mouthpiece 1 is being used and is arranged side-on to a flow region as viewed by the user. The upper support surface 5 is configured to cooperate with the occlusal surface of an upper molar 14. The lower form fitting device 4 has one lower support surface 8 which points downwards when the mouthpiece 1 is being used and is arranged side-on to a flow region as viewed by the user. The lower support surface 8 is configured to cooperate with the occlusal surface of a lower molar 14. The upper and lower support surfaces 5, 8 are dimensioned such that tilting and falling of the mouthpiece 1 can easily be prevented.

The mouthpiece 1 shown in FIG. 12 has a holding device 2 with two upper form fitting devices 3 and two lower form fitting devices 4. The mouthpiece 1 shown in FIG. 12 resembles the mouthpiece 1 shown in FIGS. 10 and 11, however there are two additional form fitting devices 3, 4 present such that the mouthpiece 1 can be held on both sides by molars 14. As a result, tilting to the side can be prevented in a simple manner.

FIGS. 13 and 14 show the embodiment of a mouthpiece 1 with holding device 2. FIG. 13 shows the upper side of the mouthpiece 1 with one upper form fitting device 3, FIG. 14 shows the underside of the mouthpiece 1 with one lower form fitting device 4.

FIG. 13 shows an upper form fitting device 3 which has two ribs. The ribs are configured to cooperate with one or a plurality of upper incisors. The ribs are suitable, in cooperation with a tooth, to prevent the mouthpiece 1 from slipping out of a mouth. Depending on how the upper and lower incisors of a user are arranged in relation to each other, it will be more comfortable for a user to support the teeth on the outer rib or on the rib which is parallel to the outer rib.

An exhalation valve 15 can also be seen in FIG. 13.

FIG. 14 shows a lower form fitting device 4 which has two ribs. The ribs are configured to cooperate with one or a plurality of lower incisors. The rib arranged on the outer edge of the mouthpiece 1 is suitable, in cooperation with a lower incisor, to prevent the mouthpiece 1 from slipping out of a mouth.

The second rib which is arranged parallel thereto is suitable to prevent tilting of the mouthpiece 1 by supporting the mouthpiece 1 against the vestibular surface of a lower incisor.

1 Mouthpiece

2 Holding device

3 Upper form fitting device

4 Lower form fitting device

5 Upper support surface

6 Oral surface of an upper incisor

7 Upper incisor

8 Lower support surface

9 Vestibular surface of a lower incisor

10 Lower incisor

11 Vestibular surface of an upper incisor

12 Elevation

13 Oral surface of a lower incisor

14 Molar

15 Exhalation valve

1. Mouthpiece for an inhaler device having an inlet, which is connectable to an inhaler device, an outlet and a holding device.

2. Mouthpiece according to claim 1, wherein the holding device has one upper form fitting device and one lower form fitting device.

3. Mouthpiece according to claim 2, wherein the upper form fitting device has one rib or one channel.

4. Mouthpiece according to claim 2, wherein the lower form fitting device has two ribs or one channel.

5. Mouthpiece according to claim 1, wherein the holding device has a plurality of lower form fitting devices.

6. Mouthpiece according to claim 1, wherein the holding device has a plurality of upper form fitting devices.

7. Mouthpiece according to claim 1, wherein the holding device includes a soft material.

8. Inhalation therapy unit with an inhaler device and a mouthpiece according to claim 1.
